(19) United States
(12) Patent Application Publication
(10) Pub. No.: US 2020/0281288 A1
(43) Pub. Date: Sep. 10, 2020

(54) ANTIMICROBIAL GARMENT TO WEAR DURING SURGERY AND AS PART OF A SHOULDER IMMOBILIZER SYSTEM AFTER SURGERY

(71) Applicant: Parasol Medical, LLC, Buffalo Grove, IL (US)

(72) Inventor: Patrick E. Eddy, Allendale, MI (US)

(73) Assignee: Parasol Medical, LLC, Buffalo Grove, IL (US)

(21) Appl. No.: 16/293,102

(22) Filed: Mar. 5, 2019

(51) Int. Cl. A41D 13/12; A61F 5/01; A61F 5/37; A41D 31/30

(52) U.S. Cl. CPC A41D 13/1245; A41D 31/305; A61F 5/3723; A61F 5/0118

(57) ABSTRACT

A garment for a person to wear during surgery comprising: an unapplied position where the garment is not applied to a person; an applied position where the garment is applied to the person; a length sufficiently long for the garment to wrap laterally around a thorax of a person and under arms of the person when the garment takes the applied position; a width sufficiently wide for the garment to cover a breast area of the thorax of the person and at least a portion of an abdomen of the person when the garment takes the applied position; and an exterior surface with an antimicrobial coating applied to the exterior surface; wherein, the antimicrobial coating includes a silane quaternary ammonium ion or salt thereof. The silane quaternary ammonium ion or salt thereof is one or more of: 3-(trimethoxysilyl)propyldimethyloctadecyl ammonium ion, 3-(trimethoxysilyl)propyldimethyloctadecyl ammonium chloride, 3-(trihydroxysilyl)propyldimethyloctadecyl ammonium ion, or 3-(trihydroxysilyl)propyldimethyloctadecyl ammonium chloride.

### BACKGROUND OF THE DISCLOSURE

Patients undergoing surgery can feel like they are inadequately clothed over the breast area of the patient. Surgery can require the removal of typical clothing. In addition, items in the surgical environment are preferably sterile to prevent the transmission of viruses, bacteria, and other microorganisms to the patient. Therefore, there is a need for a garment that helps patients feel more adequately clothed. In addition, there is a need for this garment to help prevent the transmission of viruses, bacteria, and other microorganisms to the patient.

After surgery, the area of the patient where the surgery occurred should be immobilized to assist in the healing process. Therefore, there is a need for a garment that assists in immobilizing the area of the patient where the surgery occurred.

### BRIEF SUMMARY OF THE DISCLOSURE

According to a first aspect of the present disclosure, a garment for a person to wear during surgery comprises: an unapplied position where the garment is not applied to a person; an applied position where the garment is applied to the person; a length sufficiently long for the garment to wrap laterally around a thorax of a person and under arms of the person when the garment takes the applied position; a width sufficiently wide for the garment to cover a breast area of the thorax of the person and at least a portion of an abdomen of the person when the garment takes the applied position; and an exterior surface with an antimicrobial coating applied to the exterior surface; wherein, the antimicrobial coating includes a silane quaternary ammonium ion or salt thereof.

Embodiments of the first aspect of the disclosure can include any one or a combination of the following features:
the silane quaternary ammonium ion or salt thereof is one or more of:
3-(trimethoxysilyl)propyldimethyloctadecyl ammonium ion,
3-(trimethoxysilyl)propyldimethyloctadecyl ammonium chloride,
3-(trihydroxysilyl)propyldimethyloctadecyl ammonium ion, or
3-(trihydroxysilyl)propyldimethyloctadecyl ammonium chloride;
   loops configured to cooperate with hooks as part of a releasably attachable hook and loop fastening system;
   the loops are disposed over the abdomen of the person when the garment takes the applied position;
   the loops are disposed to a side of the person when the garment takes the applied position; and
   the loops are further disposed over the abdomen of the person when the garment takes the applied position.

According to a second aspect of the present disclosure, a shoulder immobilizer system comprises: (A) a garment comprising: an unapplied position where the garment is not applied to a person; an applied position where the garment is applied to the person; a length sufficiently long for the garment to wrap laterally around a thorax of a person and under arms of the person when the garment takes the applied position; a width sufficiently wide for the garment to cover a breast area of the thorax of the person and at least a portion of an abdomen of the person when the garment takes the applied position; loops configured to cooperate with hooks as part of a releasably attachable hook and loop fastening system; and an exterior surface with an antimicrobial coating applied to the exterior surface; wherein, the antimicrobial coating includes a silane quaternary ammonium ion or salt thereof; and (B) a first strap comprising: an unapplied position where the first strap is not applied to the person; an applied position where the strap is applied to the person; a first side; a second side; a first end, a second end, a middle portion between the first end and the second end, and a length defined as a distance between the first end and the second end; hooks disposed at the first side, at the middle portion; and hooks disposed at the second side at the first end; wherein, in the applied position, the hooks of the first strap disposed at the first side, at the middle portion, are releasably attached to the loops of the garment at the side of the person; wherein, in the applied position, the second end of the first strap is wrapped around an arm of the person; and wherein, in the applied position, the hooks of the first end of the first strap are releasably attached to the loops of the garment at the back of the person, and the first strap compresses the arm of the person toward the side of the person.

Embodiments of the second aspect of the disclosure can include any one or a combination of the following features:
a second strap comprising: a first side; a second side; a first end and a second end, a middle portion between the first end and the second end, and a length defined as a distance between the first end and the second end; hooks disposed at the first side, at the first end; loops disposed at the second side, at the second end; hooks disposed at the second side, at the middle portion; and an applied position where the second strap is applied to the person; wherein, in the applied position, the second strap is wrapped length-wise around a wrist of the person, and the hooks of the second strap that are disposed at the first end, at the first side, are releasably attached to the loops of the second strap that are disposed at the second end, at the second side; and wherein, in the applied position, the hooks disposed at the second side, at the middle portion, are releasably attached to the loops of the garment at the abdomen of the person;
the first strap further comprising: an exterior surface with an antimicrobial coating applied to the exterior surface;
the antimicrobial coating of the first strap includes a silane quaternary ammonium ion or salt thereof;
the first strap further comprises: an exterior surface with an antimicrobial coating applied to the exterior surface;
the second strap further comprising: an exterior surface with an antimicrobial coating applied to the exterior surface;
the antimicrobial coating of the first strap and the second strap include a silane quaternary ammonium ion or salt thereof; and
the silane quaternary ammonium ion or salt thereof is one or more of:
3-(trimethoxysilyl)propyldimethyloctadecyl ammonium ion,
3-(trimethoxysilyl)propyldimethyloctadecyl ammonium chloride,
3-(trihydroxysilyl)propyldimethyloctadecyl ammonium ion, or
3-(trihydroxysilyl)propyldimethyloctadecyl ammonium chloride.

According to a third aspect of the present disclosure, a shoulder immobilizer system comprises: one or more straps; and a garment, worn during a surgical procedure performed on a shoulder of a person, that covers a breast area of the person, and when used in combination with the one or more straps, immobilizes the shoulder of the person; and the garment includes an exterior surface with an antimicrobial coating applied to the exterior surface; wherein, the antimicrobial coating includes a silane quaternary ammonium ion or salt thereof.

Embodiments of the third aspect of the disclosure can include any one or a combination of the following features:
the garment comprises: a first end and a second end along a length; a first side and a second side; a width that is less than the length; and an applied position where the length of the garment is wrapped around a thorax of a person, and the second side faces the breast area of the person, with the first end releasably attached to the first side;
one of the one or more straps wraps around an arm of the person and restricts movement of the arm by releasably attaching to the garment, and the strap further includes an exterior surface with an antimicrobial coating applied to the exterior surface;
the antimicrobial coating of the strap includes a silane quaternary ammonium ion or salt thereof; and
the silane quaternary ammonium ion or salt thereof is one or more of:
3-(trimethoxysilyl)propyldimethyloctadecyl ammonium ion,
3-(trimethoxysilyl)propyldimethyloctadecyl ammonium chloride,
3-(trihydroxysilyl)propyldimethyloctadecyl ammonium ion, or
3-(trihydroxysilyl)propyldimethyloctadecyl ammonium chloride.

These and other features, advantages, and objects of the present disclosure will be further understood and appreciated by those skilled in the art by reference to the following specification, claims, and appended drawings.

### BRIEF DESCRIPTION OF THE DRAWINGS

FIG. 1 is an overhead view of a garment in an unapplied position not applied to a person, illustrating an exterior surface and an antimicrobial coating applied to the exterior surface;

FIG. 2 is a perspective view of the garment of FIG. 1, illustrating a first end having snaps and a second end having snap receivers to receive the snaps of the first end, in order to apply the garment to a person;

FIG. 3 is a rear perspective view of the garment being applied to a person, illustrating the garment being positioned around a thorax of the person and under arms of the person;

FIG. 4 is a front perspective view of the garment of FIG. 1 in an applied position applied to the person of FIG. 3, illustrating the snap receivers having received the snaps and the garment covering a breast area of the person and extending down from the thorax to cover a portion of the abdomen of the person;

FIG. 5 is an overhead view of a shoulder immobilizer system that includes the garment of FIG. 1 and a first strap and a second strap;

FIG. 6 is a perspective view of the first strap of FIG. 5, illustrating an exterior surface and the antimicrobial coating applied to the exterior surface;

FIG. 7 is a front perspective view of the first strap of FIG. 5 being applied to the person wearing the garment of FIG. 1, illustrating hooks of the first strap releasably attached to loops of the garment at a side of the person;

FIG. 8 is a front perspective view of the first strap being applied to the person and continuing a sequence starting with FIG. 7, illustrating a second end of the first strap being wrapped around the arm of the person;

FIG. 9 is a side perspective view of the first strap being applied to the person and continuing the sequence of FIGS. 7 and 8, illustrating a first end of the first strap being pulled over the arm of the person and rearward over the back of the person, securing the second end in place over the arm, and hooks of the first strap releasably attaching to loops of the garment at the back of the person to place the first strap in an applied position applied to the person;

FIG. 10 is an overhead view of the second strap of FIG. 5, illustrating an exterior surface and the antimicrobial coating applied to the exterior surface, and also illustrating hooks disposed at a middle portion of a second side, as well as hooks disposed at a first end of a first side, and loops disposed at a second end of a second side;

FIG. 11 is a front perspective view of the second strap of FIG. 5 being applied to the person already wearing the first strap and the garment, illustrating the second strap being wrapped around a wrist of the person and the hooks of the first side being releasably coupled to the loops of the second side;

FIG. 12 is a front perspective view of the second strap being applied to the person and continuing the sequence starting with FIG. 11, illustrating the hooks of the first side releasably coupled to the loops of the second side, and hooks at a middle portion of the second side being releasably attached to loops of the garment disposed near the abdomen of the person; and

FIG. 13 is a front perspective view of the second strap in an applied position applied to the person, and the shoulder immobilizer system immobilizing the shoulder of the person, and the antimicrobial coating applied to the exterior surfaces of the garment, the first strap, and the second strap to reduce the risk of transmission of microbes and viruses to the person post-surgery.

### DETAILED DESCRIPTION

For purposes of description herein, it is to be understood that the specific devices and processes illustrated in the attached drawings, and described in the following specification are simply exemplary embodiments of the inventive concepts defined in the appended claims. Hence, physical characteristics relating to the embodiments disclosed herein are not to be considered as limiting, unless the claims expressly state otherwise.

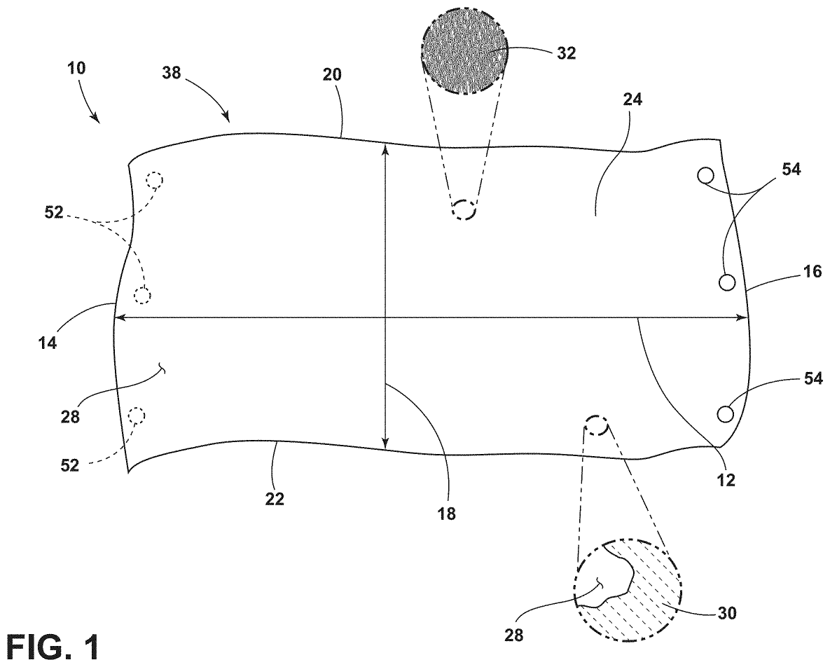

Referring to FIG. 1, a garment 10 is illustrated. The garment 10 includes a length 12 that extends from a first end 14 to a second end 16. The garment 10 further includes a width 18 that is generally orthogonal to the length 12 and extends from a third end 20 to a fourth end 22. In the illustrated embodiment, the width 18 is less than the length 12, that is, the measurement value for the width 18 is numerally less than the measurement value for the length 12. However, the width 18 need not be less than the length 12. The garment 10 further includes a first side 24 and a second side 26 that faces in a general opposite direction than the first side 24. The garment 10 further includes an exterior surface 28, which is present on both the first side 24 and the second side 26. The garment 10 further includes an antimicrobial coating 30 applied to the exterior surface 28. The antimicrobial coating 30 need not be applied to the entirety of the exterior surface 28 but can be. The garment 10 further includes loops 32, which as detailed below, as part of a releasably attachable hook and loop fastening system. The loops 32 are disposed at the first side 24 and can be disposed along the second side 26 as well.

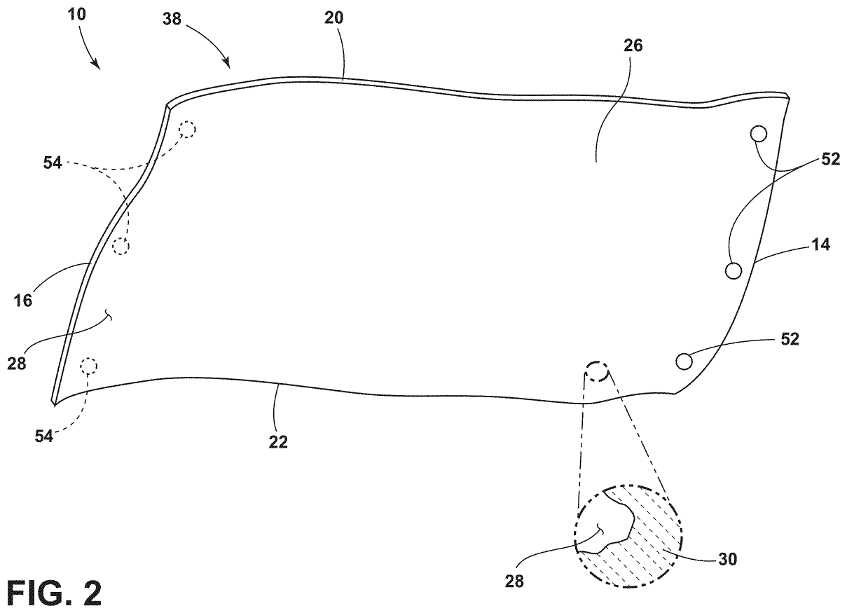

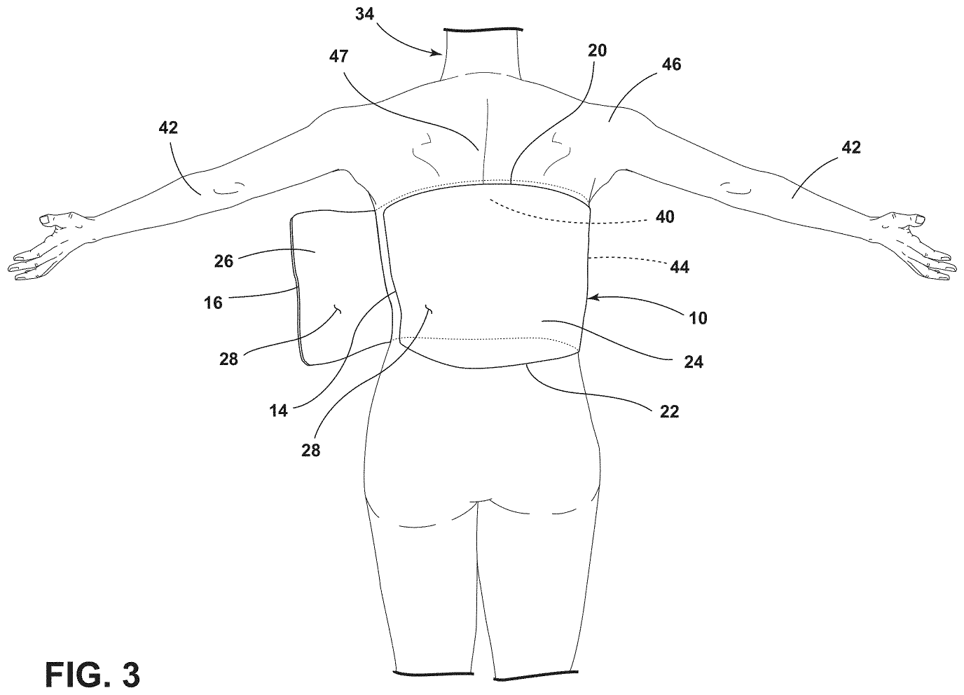

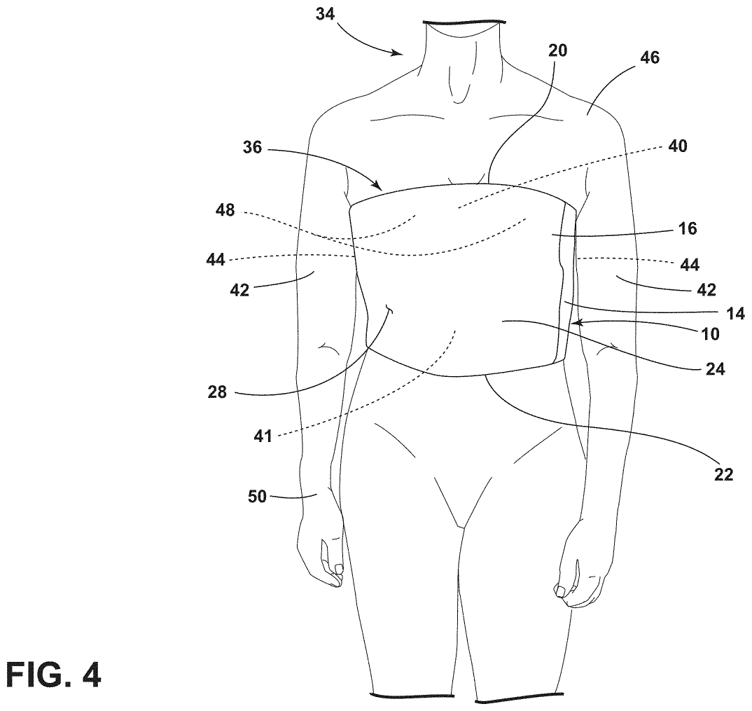

Referring now additionally to FIGS. 3 and 4, the garment 10 is illustrated being applied to a person 34 in FIG. 3 and then finally in FIG. 4 in an applied position 36 where the garment 10 is applied to the person 34. In contrast, the garment 10 is illustrated in an unapplied position 38, where the garment 10 is not applied to a person 34, in FIGS. 1 and 2. The person 34 has a thorax 40, an abdomen 41 below the thorax 40, an arm 42, a side 44, a shoulder 46, a back 47, a breast area 48 at the thorax 40, and a wrist 50 at the arm 42. The length 12 of the garment 10 is sufficiently long for the garment 10 to wrap laterally around the thorax 40 of a person 34 and under the arm 42 of the person 34, when the garment 10 takes the applied position 36. To secure the garment 10 in the applied position 36, the garment 10 can include any type of fastening system. In the illustrated embodiment, the garment 10 includes snaps 52 disposed at the first end 14 and snap receivers 54 disposed at the second end 16. The garment 10 can be placed with the second side 26 facing the person 34 and the length 12 of the garment 10 laterally across the thorax 40 of the person 34, then the garment 10 is wrapped around the person 34 until the snap receivers 54 of the second end 16 can releasably receive the snaps 52 of the first end 14, thus securing and applying the garment 10 to the person 34. When so snapped, the first end 14 is releasably attached to the first side 24, because the snap receivers 54 are available to receive at the first side 24. The width 18 is sufficiently wide for the garment 10 to cover the breast area 48 of the thorax 40 of the person 34, when the garment 10 is in the applied position 36, with the second side 26 of the garment 10 facing the breast area 48. In addition, in the illustrated embodiment, the width 18 is sufficiently wide to cover at least a portion of the abdomen 41 of the person 34, when the garment 10 is in the applied position 36. In other words, in the applied position 36, the garment 10 covers the breast area 48 of the person 34 and at least a portion of the abdomen 41 of the person 34.

The garment 10 thus helps the person 34 (the patient) feel more adequately clothed because the garment 10 covers the breast area 48 of the person 34 during surgery. In addition, because the antimicrobial coating 30 is applied to the exterior surface 28 of the garment 10, the person 34 can wear the garment 10 in the surgical environment during the surgery, such as during surgery on the shoulder 46 of the person 34. Applying the antimicrobial coating 30 to the exterior surface 28 of the garment 10 imparts antimicrobial properties on the exterior surface 28 and to the garment 10 generally, which prevents or lessens the ability of the exterior surface 28 to harbor harmful microbes, viruses, bacteria, and the like and spread them to the person 34 wearing the garment 10 during surgery. The garment 10 is thus worn during a surgical procedure performed on the shoulder 46 of the person 34. During the surgical procedure, the garment 10 covers the breast area 48 of the person 34.

For reasons that will become apparent below, the loops 32 of the garment 10 are disposed over the abdomen 41 of the person 34 when the garment 10 is in the applied position 36. In addition, in the illustrated embodiment, the loops 32 are further disposed to the side 44 of the person 34 and to the back 47 of the person 34 when the garment 10 takes the applied position 36. In the illustrated embodiment, essentially the entirety of the first side 24 of the garment 10 contains the loops 32. The first side 24 of the garment 10 can be constructed from a looped fabric to provide the loops 32.

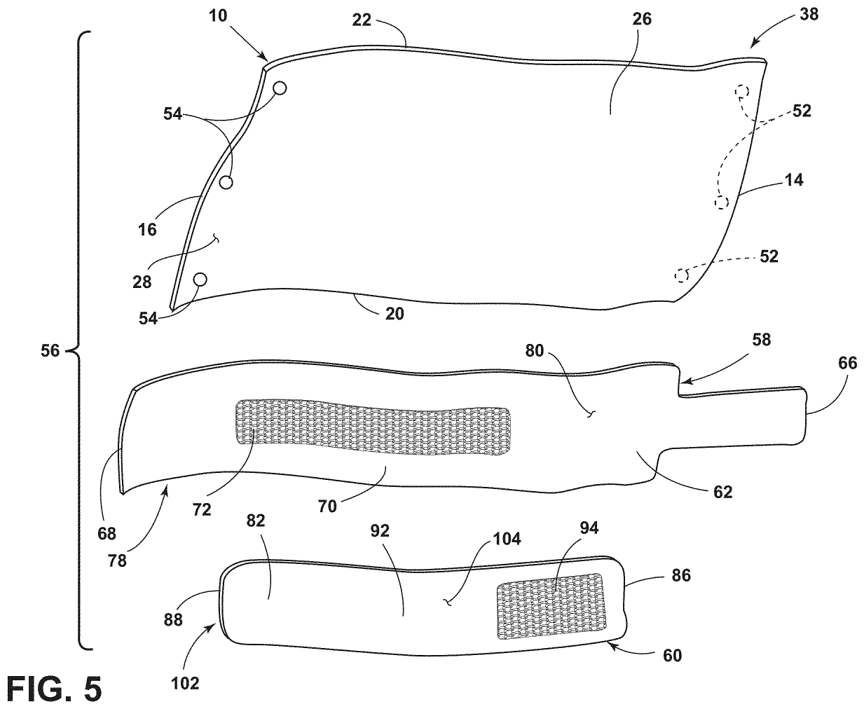

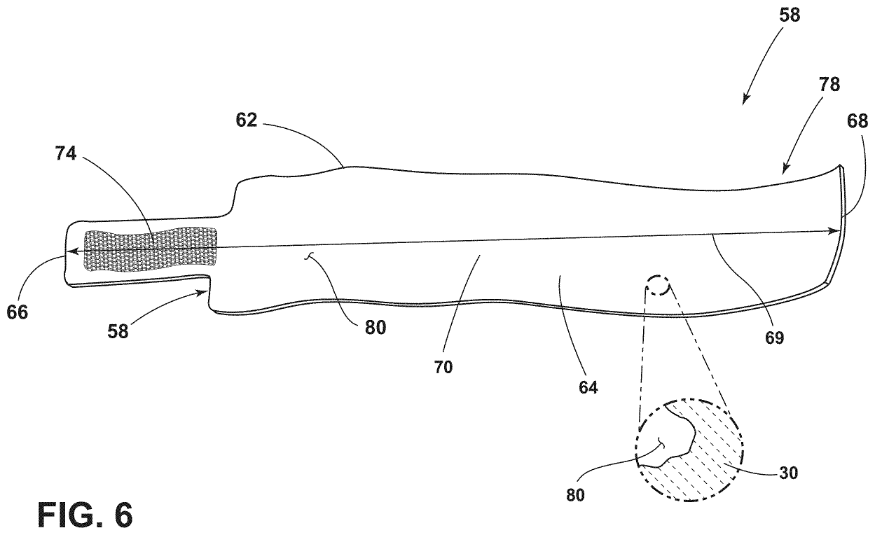

Referring now additionally to FIGS. 5 and 6, the garment 10 is a component in a shoulder immobilizer system 56, which further includes a first strap 58 and optionally a second strap 60. The first strap 58 includes a first side 62 and a second side 64 facing the general opposite direction as the first side 62. The first strap 58 further includes a first end 66 and a second end 68. The first strap 58 has a length 69 defined as the distance between the first end 66 and the second end 68. The first strap 58 further includes a middle portion 70 between the first end 66 and the second end 68. The first strap 58 further includes hooks 72, 74 that are configured to participate in a hook and loop fastening system. The hooks 72 are disposed at the first side 62, at the middle portion 70. In addition, the hooks 74 are disposed at the second side 64, at the first end 66.

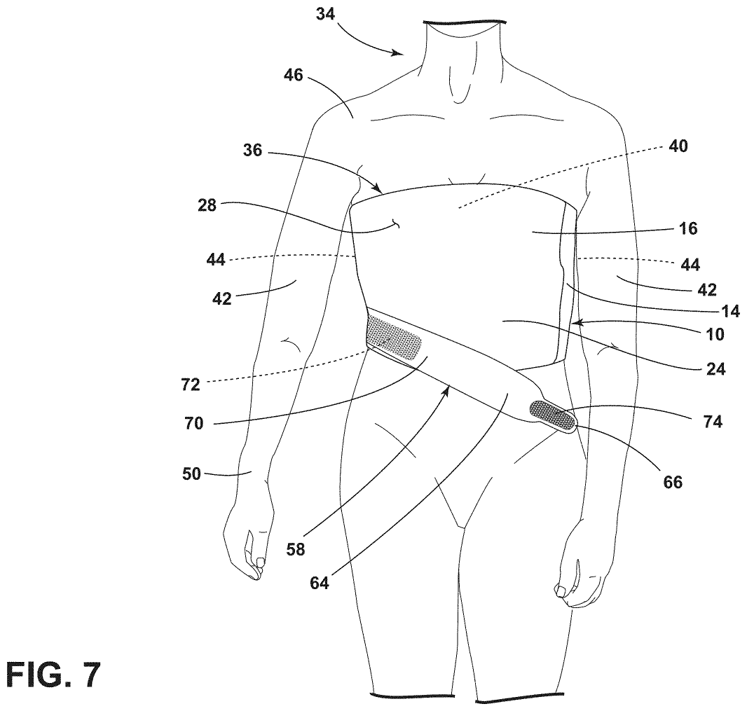

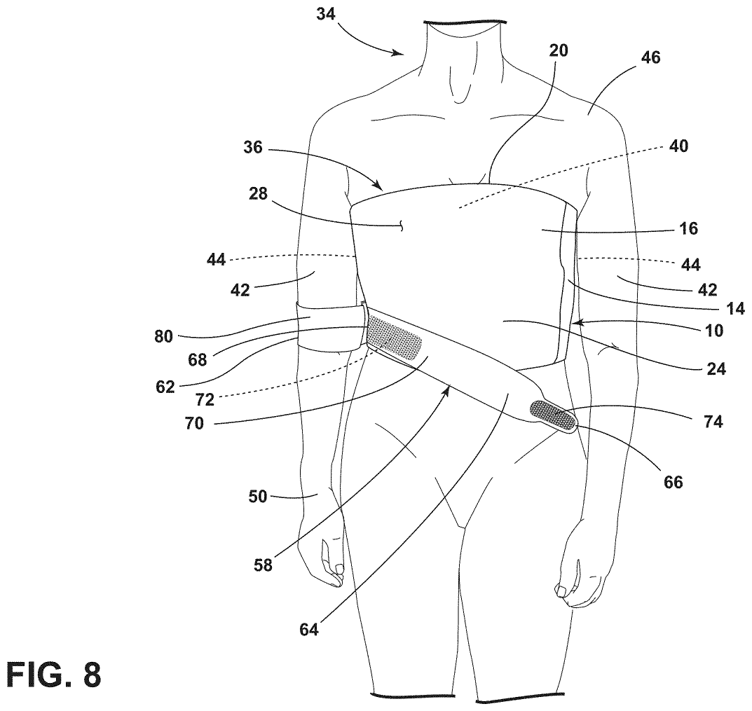

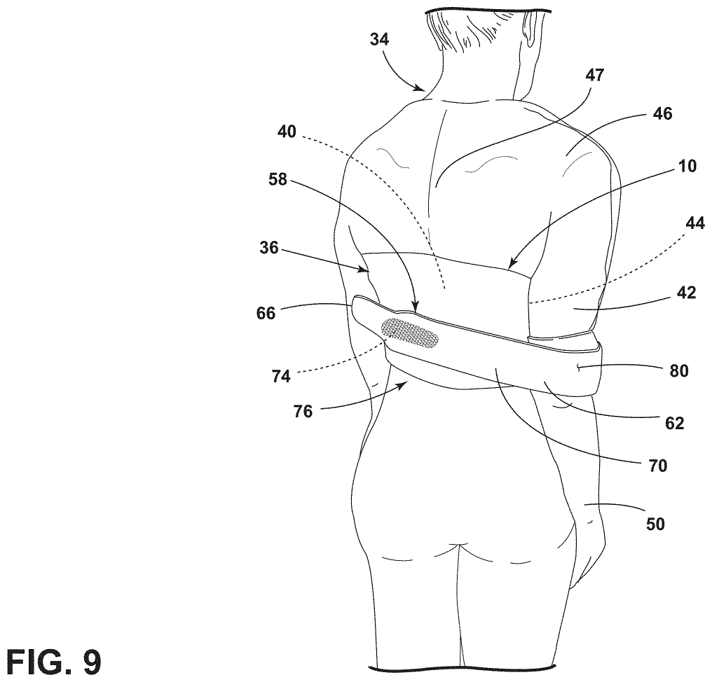

Referring now additionally to FIGS. 7-9, the first strap 58 is illustrated transitioning to an applied position 76 (FIG. 9). As illustrated in FIG. 7, the hooks 72 of the first strap 58 that are disposed at the first side 62, at the middle portion 70, are releasably attached to the loops 32 of the garment 10 that are at the side 44 of the person 34. The length 69 of the first strap 58 is generally to parallel the length 12 of the garment 10, when the first strap 58 and the garment 10 are in their respective applied positions 76, 36. As illustrated at FIG. 8, the second end 68 of the first strap 58 is then wrapped lengthwise around the arm 42 of the person 34 that is closest to the side 44 of the person 34 where hooks 72 of the first strap 58 are releasably attached to the loops 32 of the garment 10. As illustrated in FIG. 9, the hooks 74 of the first end 66 of the first strap 58 are releasably attached to the loops 32 of the garment 10 at the back 47 of the person 34. The first end 66 of the first strap 58 is pulled tightly across the back 47 of the person 34 so that the first strap 58 compresses the arm 42 of the person 34 toward, to, and against the side 44 of the person 34. The first strap 58 is now (FIG. 9) in the applied position 76 where the first strap 58 is applied to the person 34. The first strap 58 is illustrated in FIGS. 5 and 6 in an unapplied position 78 where the first strap 58 is not applied to the person 34. The first strap 58 is illustrated in FIGS. 7 and 8 transitioning from the unapplied position 78 (FIGS. 5 and 6) to the applied position 76 (FIG. 9). In the applied position 76, the first strap 58 prevents the arm 42 from pivoting about the shoulder 46 laterally, forward, or rearward. Like the garment 10, the first strap 58 further includes an exterior surface 80. The antimicrobial coating 30 can be additionally applied to the exterior surface 80 of the first strap 58.

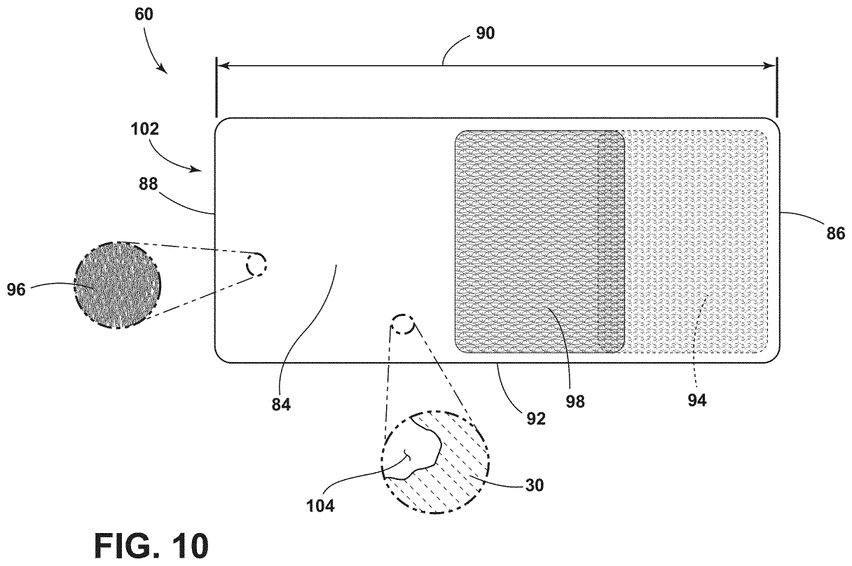

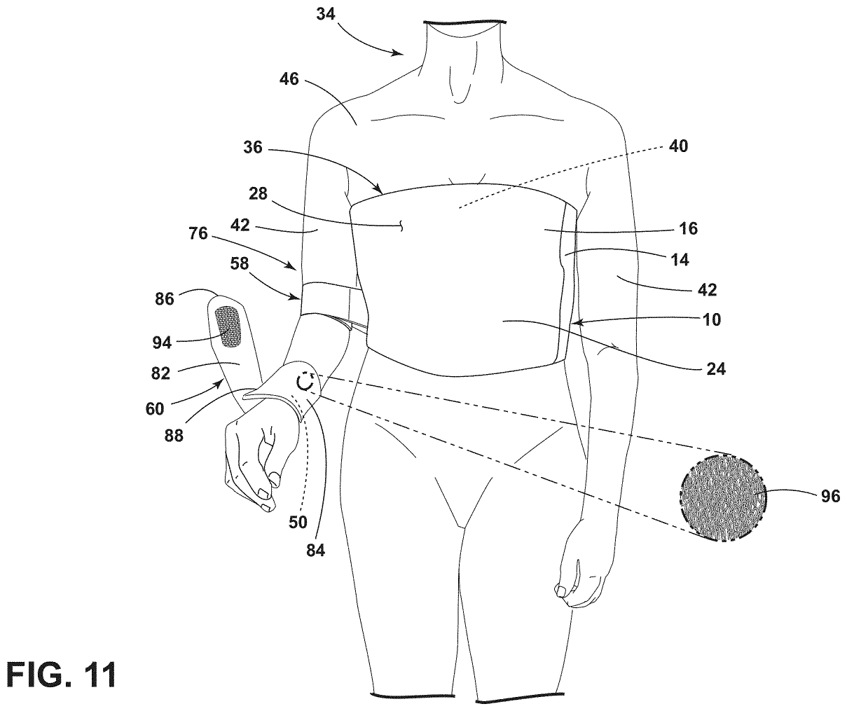

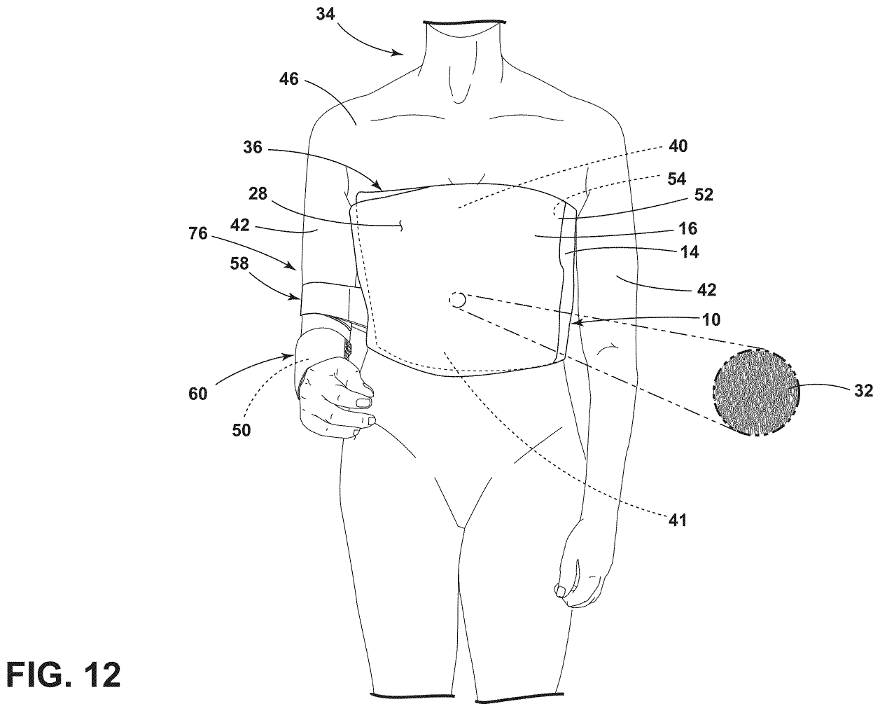

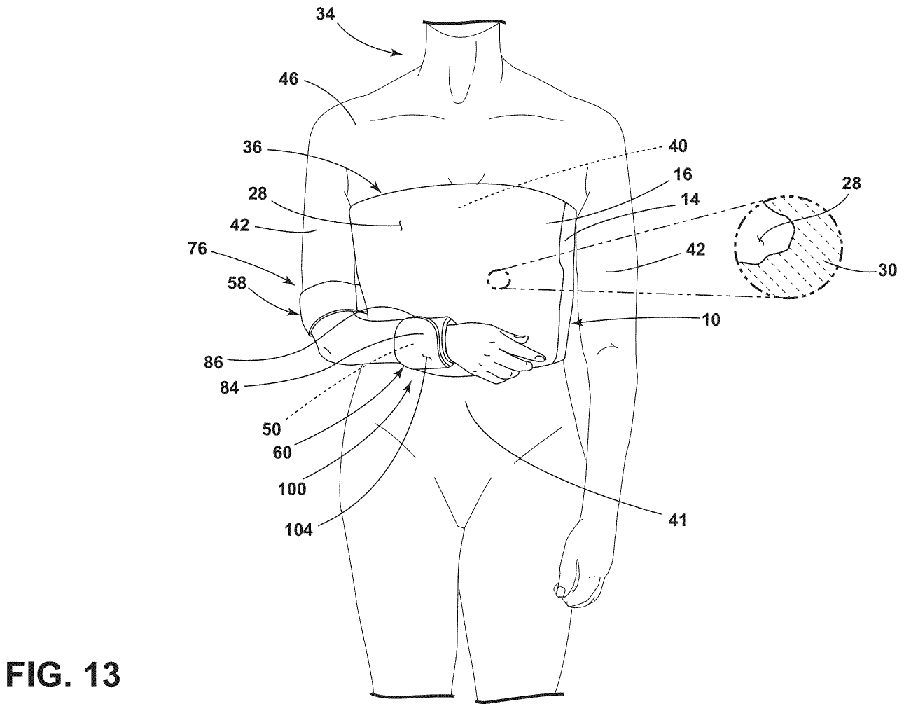

Referring now back to FIG. 5, and additionally to FIGS. 10-12, the second strap 60 includes a first side 82 and a second side 84 that faces in the general opposite direction as the first side 82. The second strap 60 further includes a first end 86 and a second end 88, and a length 90 defined as the distance between the first end 86 and the second end 88. A middle portion 92 is between the first end 86 and the second end 88. The second strap 60 further includes hooks 94 disposed at the first side 82, at the first end 86. The second strap 60 further includes loops 96 disposed at the second side 84, at the second end 88. The second strap 60 further includes hooks 98 disposed at the second side 84, at the middle portion 92. As illustrated in FIG. 11, the second strap 60 is wrapped length-wise around the wrist 50 of the person 34. As illustrated in FIG. 12, the hooks 94 that are disposed at the first end 86, at the first side 82, are then releasably attached to the loops 96 of the second strap 60 that are disposed at the second end 88, at the second side 84. As illustrated in FIG. 13, the hooks 98 that are then disposed at the second side 84, at the middle portion 92, are releasably attached to the loops 32 of the garment 10 at the abdomen 41 of the person 34. As a consequence, the second strap 60 is in an applied position 100 where the second strap 60 is applied to the person 34. The second strap 60 is illustrated in an unapplied position 102, where the second strap 60 is not applied to the person 34, in FIGS. 5 and 10. The second strap 60 is illustrated in the applied position 100 in FIG. 13. The second strap 60 is illustrated transitioning from the unapplied position 102 to the applied position 100 in FIGS. 11 and 12. Like the garment 10 and the first strap 58, the second strap 60 further includes an exterior surface 104. The antimicrobial coating 30 is applied to the exterior surface 104. The second strap 60 further immobilizes the shoulder 46 of the person 34 by preventing rotational movement of the arm 42 about the shoulder 46.

Accordingly, the shoulder immobilizer system 56 includes the garment 10 described above, and one or more straps, such as the first strap 58 and the second strap 60. The straps 58, 60 are able to releasably attach to the garment 10, allowing the garment 10 and the one or more straps 58, 60 to immobilize the shoulder 46 of the person 34, such as after surgery. The straps 58, 60 wrap around the arm 42 of the person 34 that underwent surgery of the shoulder 46 to restrict movement of the arm 42 by releasably attaching to the garment 10.

The antimicrobial coating 30 applied to the exterior surfaces 28, 80, 104 of the garment 10, the first strap 58, and the second strap 60, include a silane quaternary ammonium ion or salt thereof. Preferred silane quaternary ammonium ions or salts thereof include
3-(trimethoxysilyl)propyldimethyloctadecyl ammonium ion,
3-(trimethoxysilyl)propyldimethyloctadecyl ammonium chloride,
3-(trihydroxysilyl)propyldimethyloctadecyl ammonium ion, or
3-(trihydroxysilyl)propyldimethyloctadecyl ammonium chloride. The antimicrobial coating 30 imparts antimicrobial properties to the garment 10 and the shoulder immobilizer system 56 generally including the first strap 58 and the second strap 60. The garment 10, the first strap 58, and the second strap 60 thus reduce the likelihood of transmitting microbes to the shoulder 46 where the surgery occurred and thus reduce the risk of infection post-surgery. Because the antimicrobial coating 30 is applied to the exterior surfaces 28, 80, 104 of the garment 10, the first strap 58, and the second strap 60, those articles are much less likely to harbor bacteria, viruses, and other microorganisms that could harm the person 34 after surgery.

To apply the antimicrobial coating 30 onto the exterior surfaces 28, 80, 104, a solution including the silane quaternary ammonium ion or salt thereof is applied to the exterior surfaces 28, 80, 104. In addition to the silane quaternary ammonium ion or salt thereof, the solution can further include a solvent. A preferred solvent is isopropyl alcohol. After application of the solution to the exterior surfaces 28, 80, 104 the solvent, if utilized, generally evaporates, which leaves the silane quaternary ammonium ion or salt thereof applied to the exterior surfaces 28, 80, 104. Again, preferred silane quaternary ammonium ions or salts thereof include
3-(trimethoxysilyl)propyldimethyloctadecyl ammonium ion,
3-(trimethoxysilyl)propyldimethyloctadecyl ammonium chloride,
3-(trihydroxysilyl)propyldimethyloctadecyl ammonium ion, or
3-(trihydroxysilyl)propyldimethyloctadecyl ammonium chloride. The solution can be applied to the exterior surfaces 28, 80, 104 via any application method, including electrostatic spraying, dipping, and coating.

The silane quaternary ammonium ion or salt thereof can comprise between 0.1 percent and 10 percent by weight of the solution. More preferably, the silane quaternary ammonium ion or salt thereof can comprise between 0.75 percent and 5 percent by weight of the solution. Even more preferably, the silane quaternary ammonium ion or salt thereof can comprise between 1.9 percent and 2.1 percent by weight of the solution.

As for the isopropyl alcohol, the isopropyl alcohol can comprise between 30 percent to 90 percent by weight of the solution. More preferably, the isopropyl alcohol can comprise between 55 percent and 65 percent by weight of the solution. An example preferable solution comprises (by weight) 60.0 percent isopropyl alcohol, 2.02 percent 3-(trimethoxysilyl)propyldimethyloctadecyl ammonium chloride, and 34.19 percent deionized water.

It is to be understood that variations and modifications can be made on the aforementioned structure without departing from the concepts of the present disclosure, and further it is to be understood that such concepts are intended to be covered by the following claims unless these claims by their language expressly state otherwise.

What is claimed is:

1. A garment for a person to wear during surgery comprising:
an unapplied position where the garment is not applied to a person;
an applied position where the garment is applied to the person;
a length sufficiently long for the garment to wrap laterally around a thorax of a person and under arms of the person when the garment takes the applied position;
a width sufficiently wide for the garment to cover a breast area of the thorax of the person and at least a portion of an abdomen of the person when the garment takes the applied position; and
an exterior surface with an antimicrobial coating applied to the exterior surface;
wherein, the antimicrobial coating includes a silane quaternary ammonium ion or salt thereof.

2. The garment of claim 1,
wherein, the silane quaternary ammonium ion or salt thereof is one or more of:
3-(trimethoxysilyl)propyldimethyloctadecyl ammonium ion,
3-(trimethoxysilyl)propyldimethyloctadecyl ammonium chloride,
3-(trihydroxysilyl)propyldimethyloctadecyl ammonium ion, or
3-(trihydroxysilyl)propyldimethyloctadecyl ammonium chloride.

3. The garment of claim 1 further comprising:
loops configured to cooperate with hooks as part of a releasably attachable hook and loop fastening system.

4. The garment of claim 3,
wherein, the loops are disposed over the abdomen of the person when the garment takes the applied position.

5. The garment of claim 3,
wherein, the loops are disposed to a side of the person when the garment takes the applied position.

6. The garment of claim 5,
wherein, the loops are further disposed over the abdomen of the person when the garment takes the applied position.

7. A shoulder immobilizer system comprising:
a garment comprising:
an unapplied position where the garment is not applied to a person;
an applied position where the garment is applied to the person;
a length sufficiently long for the garment to wrap laterally around a thorax of a person and under arms of the person when the garment takes the applied position;
a width sufficiently wide for the garment to cover a breast area of the thorax of the person and at least a portion of an abdomen of the person when the garment takes the applied position;
loops configured to cooperate with hooks as part of a releasably attachable hook and loop fastening system; and
an exterior surface with an antimicrobial coating applied to the exterior surface;
wherein, the antimicrobial coating includes a silane quaternary ammonium ion or salt thereof; and
a first strap comprising:
an unapplied position where the first strap is not applied to the person;
an applied position where the first strap is applied to the person;
a first side;
a second side;
a first end, a second end, a middle portion between the first end and the second end, and a length defined as a distance between the first end and the second end;
hooks disposed at the first side, at the middle portion; and
hooks disposed at the second side at the first end;
wherein, in the applied position, the hooks of the first strap disposed at the first side, the middle portion, are releasably attached to the loops of the garment at the side of the person;
wherein, in the applied position, the second end of the first strap is wrapped around an arm of the person; and
wherein, in the applied position, the hooks of the first end of the first strap are releasably attached to the loops of the garment at the back of the person, and the first strap compresses the arm of the person toward the side of the person.

8. The shoulder immobilizer system of claim 7 further comprising:
a second strap comprising:
a first side;
a second side;
a first end and a second end, a middle portion between the first end and the second end, and a length defined as a distance between the first end and the second end;
hooks disposed at the first side, at the first end;
loops disposed at the second side, at the second end;
hooks disposed at the second side, at the middle portion; and
an applied position where the second strap is applied to the person;
wherein, in the applied position, the second strap is wrapped length-wise around a wrist of the person, and the hooks of the second strap that are disposed at the first end, at the first side, are releasably attached to the loops of the second strap that are disposed at the second end, at the second side; and
wherein, in the applied position, the hooks disposed at the second side, at the middle portion, are releasably attached to the loops of the garment at the abdomen of the person.

9. The shoulder immobilizer system of claim 7,
the first strap further comprising: an exterior surface an exterior surface with an antimicrobial coating applied to the exterior surface;
wherein, the antimicrobial coating of the first strap includes a silane quaternary ammonium ion or salt thereof.

10. The shoulder immobilizer system of claim 8,
the first strap further comprising: an exterior surface an exterior surface with an antimicrobial coating applied to the exterior surface; and
the second strap further comprising: an exterior surface an exterior surface with an antimicrobial coating applied to the exterior surface;
wherein, the antimicrobial coating of the first strap and the second strap include a silane quaternary ammonium ion or salt thereof.

11. The shoulder immobilizer system of claim 7,
wherein, the silane quaternary ammonium ion or salt thereof is one or more of:
3-(trimethoxysilyl)propyldimethyloctadecyl ammonium ion,
3-(trimethoxysilyl)propyldimethyloctadecyl ammonium chloride,
3-(trihydroxysilyl)propyldimethyloctadecyl ammonium ion, or
3-(trihydroxysilyl)propyldimethyloctadecyl ammonium chloride.

12. The shoulder immobilizer system of claim 8,
wherein, the silane quaternary ammonium ion or salt thereof is one or more of:
3-(trimethoxysilyl)propyldimethyloctadecyl ammonium ion,
3-(trimethoxysilyl)propyldimethyloctadecyl ammonium chloride,
3-(trihydroxysilyl)propyldimethyloctadecyl ammonium ion, or
3-(trihydroxysilyl)propyldimethyloctadecyl ammonium chloride.

13. The shoulder immobilizer system of claim 9,
wherein, the silane quaternary ammonium ion or salt thereof is one or more of:
3-(trimethoxysilyl)propyldimethyloctadecyl ammonium ion,
3-(trimethoxysilyl)propyldimethyloctadecyl ammonium chloride,
3-(trihydroxysilyl)propyldimethyloctadecyl ammonium ion, or
3-(trihydroxysilyl)propyldimethyloctadecyl ammonium chloride.

14. The shoulder immobilizer system of claim 10,
wherein, the silane quaternary ammonium ion or salt thereof is one or more of:
3-(trimethoxysilyl)propyldimethyloctadecyl ammonium ion,
3-(trimethoxysilyl)propyldimethyloctadecyl ammonium chloride,
3-(trihydroxysilyl)propyldimethyloctadecyl ammonium ion, or
3-(trihydroxysilyl)propyldimethyloctadecyl ammonium chloride.

15. A shoulder immobilizer system comprising:
one or more straps; and
a garment, worn during a surgical procedure performed on a shoulder of a person, that covers a breast area the person, and when used in combination with the one or more straps, immobilizes the shoulder of the person; and
the garment includes an exterior surface with an antimicrobial coating applied to the exterior surface;
wherein, the antimicrobial coating includes a silane quaternary ammonium ion or salt thereof.

16. The shoulder immobilizer system of claim 15,
the garment comprising:
a first end and a second end along a length;
a first side and a second side;
a width that is less than the length; and
an applied position where the length of the garment is wrapped around a thorax of a person, and the second side faces the breast area of the person, with the first end releasably attached to the first side.

17. The shoulder immobilizer system of claim 16,
wherein, one of the one or more straps wraps around an arm of the person and restricts movement of the arm by releasably attaching to the garment, and the strap further includes an exterior surface with an antimicrobial coating applied to the exterior surface; and
wherein, the antimicrobial coating of the strap includes a silane quaternary ammonium ion or salt thereof.

18. The shoulder immobilizer system of claim 15,
wherein, the silane quaternary ammonium ion or salt thereof is one or more of:
3-(trimethoxysilyl)propyldimethyloctadecyl ammonium ion,
3-(trimethoxysilyl)propyldimethyloctadecyl ammonium chloride,
3-(trihydroxysilyl)propyldimethyloctadecyl ammonium ion, or
3-(trihydroxysilyl)propyldimethyloctadecyl ammonium chloride.

19. The shoulder immobilizer system of claim 16,
wherein, the silane quaternary ammonium ion or salt thereof is one or more of:
3-(trimethoxysilyl)propyldimethyloctadecyl ammonium ion,
3-(trimethoxysilyl)propyldimethyloctadecyl ammonium chloride,
3-(trihydroxysilyl)propyldimethyloctadecyl ammonium ion, or
3-(trihydroxysilyl)propyldimethyloctadecyl ammonium chloride.

20. The shoulder immobilizer system of claim 17,
wherein, the silane quaternary ammonium ion or salt thereof is one or more of:
3-(trimethoxysilyl)propyldimethyloctadecyl ammonium ion,
3-(trimethoxysilyl)propyldimethyloctadecyl ammonium chloride,
3-(trihydroxysilyl)propyldimethyloctadecyl ammonium ion, or
3-(trihydroxysilyl)propyldimethyloctadecyl ammonium chloride.
